(19) United States
(12) Patent Application Publication
(10) Pub. No.: US 2008/0109064 A1
(43) Pub. Date: May 8, 2008

(54) Methods and Devices for Biological Fixation of Stent Grafts

(75) Inventor: Josiah Wilcox, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/556,459

(22) Filed: Nov. 3, 2006

(51) Int. Cl. A61F 2/06

(52) U.S. Cl. 623/1.13

(57) ABSTRACT

Methods and devices are provided to contribute to improved stent graft fixation within vessels at treatment sites. Improved stent graft fixation within vessels at treatment sites is provided by providing stent grafts and methods of making and using stent grafts with bare metal portions having a coating comprising a polymeric material and a cell growth promoting factor.

### FIELD OF THE INVENTION

Methods and devices for preventing stent graft migration and endoleak using site-specific cell growth promoting factor-containing compositions are disclosed. Specifically, stent grafts and methods for coating the metal portions of stent grafts with substances comprising biocompatible polymers and cell growth promoting factors are provided.

### BACKGROUND OF THE INVENTION

Stent grafts have been developed to treat abnormalities of the vascular system. Stent grafts are primarily used to treat aneurysms of the vascular system and have also emerged as a treatment for a related condition, acute blunt aortic injury, where trauma causes damage to an artery.

Aneurysms arise when a thinning, weakening section of a vessel wall dilates and balloons out. Aortic aneurysms (both abdominal and thoracic) are treated when the vessel wall expands to more than 150% of its normal diameter. These dilated and weakened sections of vessel walls can burst, causing an estimated 32,000 deaths in the United States each year. Additionally, aneurysm deaths are suspected of being underreported because sudden unexplained deaths, about 450,000 in the United States alone, are often simply misdiagnosed as heart attacks or strokes while many of them may be due to aneurysms.

U.S. surgeons treat approximately 50,000 abdominal aortic aneurysms each year, typically by replacing the abnormal section of vessel with a plastic or fabric graft in an open surgical procedure. A less-invasive procedure that has more recently been used is the placement of a stent graft at the aneurysm site. Stent grafts are tubular devices that span the aneurysm site to provide support without replacing a section of the vessel. The stent graft, when placed within a vessel at an aneurysm site, acts as a barrier between blood flow and the weakened wall of a vessel, thereby decreasing pressure on the damaged portion of the vessel. This less invasive approach to treat aneurysms decreases the morbidity seen with conventional aneurysm repair. Additionally, patients whose multiple medical comorbidities make them excessively high risk for conventional aneurysm repair are candidates for stent grafting.

While stent grafts represent improvements over previously-used vessel treatment options, there are still risks associated with their use. The most common of these risks is migration of the stent graft due to hemodynamic forces within the vessel. Stent graft migrations can lead to endoleaks, a leaking of blood into the aneurysm sac between the outer surface of the graft and the inner lumen of the blood vessel which can increase the risk of vessel rupture. Such migrations of stent grafts are especially possible in curved portions of vessels where hemodynamic forces are asymmetrical placing uneven forces on the stent graft. Additionally, the asymmetrical hemodynamic forces can cause remodeling of an aneurysm sac which leads to increased risk of aneurysm rupture and increased endoleaks.

Based on the foregoing, one goal of treating aneurysms is to provide stent grafts that do not migrate. To achieve this goal, stent grafts with stainless steel anchoring barbs that engage the vessel wall have been developed. Additionally, endostaples that fix stent grafts more readily to the vessel wall have been developed. While these physical anchoring devices have proven to be effective in some patients, they have not sufficiently ameliorated stent graft migration associated with current treatment methods in all cases.

An additional way to reduce the risk of stent graft migration is to administer to the treatment site, either before, during or relatively soon after implantation, a cell growth promoting factor (also known in some instances as an endothelialization factor). This administration can be beneficial because, normally, the endothelial cells that make up the portion of the vessel to be treated are quiescent at the time of stent graft implantation and do not multiply. As a result, the stent graft rests against a quiescent endothelial cell layer. If cell growth promoting factors are administered immediately before, during or relatively soon after stent graft deployment and implantation, the normally quiescent endothelial cells lining the vessel wall, and in intimate contact with the stent graft, will be stimulated to proliferate. The same will occur with smooth muscle cells and fibroblasts found within the vessel wall. As these cells proliferate they can grow around the stent graft such that the device becomes physically attached to the vessel wall rather than merely resting against it. This cell growth helps to prevent stent graft migration, although it may not be successful in all circumstances. Therefore, there is still room for improvement in preventing stent graft migration.

Most stent grafts provide cell growth promoting factors on the fabric of the stent graft. Because stent graft fabric is smooth, however, this area of the graft may not provide the optimal surface to promote cell growth. The present invention, recognizing this limitation, places cell growth promoting factors on metal portions of stent grafts which can provide a more irregular surface thus promoting more secure anchoring of the stent graft.

### SUMMARY OF THE INVENTION

Embodiments according to the present invention include methods and devices that are useful in reducing the risk of implantable stent graft migration. More specifically, methods and devices that promote implantable stent graft attachment to blood vessel luminal walls are provided. One embodiment provides methods and devices useful for minimizing post-implantation stent graft migration following deployment at an aneurysmal treatment site and is also useful in preventing or minimizing post-implantation endoleak following stent-graft deployment at an aneurysmal treatment site.

Embodiments according to the present invention offer these advantages by providing cell growth promoting factors on metal portions of stent grafts which can provide a more irregular surface thus promoting more secure anchoring of the stent graft. Specifically, in one embodiment, a stent graft is provided comprising one or more exposed bare metal portions and a substance on one or more of said bare metal portions wherein said substance promotes cell growth. In one embodiment, at least one of the bare metal portions is found at the end of said stent graft.

One embodiment of the stent grafts according to the present invention is a stent graft comprising bare metal portions and a substance on the bare metal portions wherein the substance comprises a biocompatible polymer and a cell growth promoting factor. In another embodiment, the biocompatible polymer is biodegradable. In another embodiment, the biocompatible and biodegradable polymer is selected from the group consisting of polyglycolic acid, poly˜glycolic acid/poly-L-lactic acid copolymers, polycaprolactone, polyhydroxybutyrate/hydroxyvalerate copolymers, poly-L-lactide, polydioxanone, polycarbonates, and polyanhydrides.

In another embodiment of the stent grafts according to the present invention, the cell growth promoting factor is basic fibroblast growth factor.

The present invention also comprises methods. One method according to the present invention comprises a method for treating an aneurysm comprising providing a stent graft comprising one or more exposed bare metal portions and a substance on one or more of the bare metal portions wherein the substance promotes cell growth. In another embodiment of the methods at least one of the provided bare metal portions is located at the end of the stent graft. In another embodiment, the substance comprises a biocompatible polymer and a cell growth promoting factor.

In another embodiment of the methods according to the present invention, the substance is a biocompatible and biodegradable polymer. In another embodiment of the methods according to the present invention, the biocompatible and biodegradable polymer is selected from the group consisting of polyglycolic acid, poly-glycolic acid/poly-L-lactic acid copolymers, polycaprolactone, polyhydroxybutyrate/hydroxyvalerate copolymers, poly-L-lactide, polydioxanone, polycarbonates, and polyanhydrides.

In another embodiment of the methods according to the present invention, the cell growth promoting factor is basic fibroblast growth factor.

Another method according to the present invention comprises a method ofproviding a stent graft comprising one or more exposed bare metal ends and a substance on one or more of the bare metal ends wherein the substance promotes cell growth, is in the form of a polymeric material comprising a cell growth promoting factor; wherein said cell growth promoting substance comprises basic fibroblast growth factor; said polymeric material is selected from the group consisting of polyglycolic acid, poly˜glycolic acid/poly-L-lactic acid copolymers, polycaprolactone, polyhydroxybutyrate/hydroxyvalerate copolymers, poly-L-lactide, polydioxanone, polycarbonates, polyanhydrides; and positioning said stent graft at a treatment site wherein the substance contributes to the fixation of the stent graft to the vessel wall at the treatment site. In another embodiment, the treatment site is an aneurysm site.

### BRIEF DESCRIPTION OF THE DRAWINGS

FIG. 1 depicts a schematic diagram of a representative stent graft that can be used in accordance with the present invention deployed at a treatment site.

FIG. 2 depicts a distal end of an injection and delivery catheter that can be used in accordance with the present invention.

FIG. 3 depicts a close-up view of the distal portion of a representative stent graft.

### DEFINITION OF TERMS

Prior to setting forth embodiments according to the present invention, it may be helpful to an understanding thereof to set forth definitions of certain terms that will be used hereinafter. Unless otherwise explained, all technical and scientific terms used herein have the same meaning as commonly understood by one of ordinary skill in the art to which this invention belongs. The singular terms “a,” “an,” and “the” include plural referents unless context clearly indicates otherwise. Similarly, the word “or” is intended to include “and” unless the context clearly indicates otherwise. The term “comprises” means “includes.”

Aortic aneurysm: As used herein “aortic aneurysm” shall include a weak section of an animal's aorta. As used herein, an “aortic aneurysm” includes, without limitation, abdominal and thoracic aneurysms.

Biocompatible: As used herein “biocompatible” refers to any material that does not cause injury or death to the animal or induce an adverse reaction in an animal when placed in intimate contact with the animal's tissues. Adverse reactions include, without limitation, inflammation, infection, fibrotic tissue formation, cell death, embolizations and/or thrombosis.

Bioactive Material: As used herein, “bioactive material(s)” shall include any, drug, compound, substance or composition that creates a physiological and/or biological effect in an animal. Non-limiting examples of bioactive materials include small molecules, peptides, proteins, hormones, DNA or RNA fragments, genes, cells, genetically-modified cells, cell growth promoting factors, matrix metalloproteinase inhibitors, autologous platelet gel, platelet rich plasma, either inactivated or activated, other natural and synthetic gels, such as, without limitation, alginates, collagens, and hyaluronic acid, polyethylene oxide, polyethylene glycol, and polyesters, as well as combinations of these bioactive materials.

Cell Growth Promoting Factors: As used herein, “cell growth promoting factors” or “cell growth promoting compositions” shall include any bioactive material having a growth promoting effect on vascular cells. Non-limiting examples of cell growth promoting factors include vascular endothelial growth factor (VEGF), platelet-derived growth factor (PDGF), platelet-derived epidermal growth factor (PDEGF), basic fibroblast growth factor (bFGF), acidic fibroblast growth factor (aFGF), transforming growth factor-beta (TGF-β), platelet-derived angiogenesis growth factor (PDAF) and autologous platelet gel (APG) including platelet rich plasma (PRP), platelet poor plasma (PPP) and thrombin.

Endoleak: As used herein, “endoleak” refers to the presence of blood flow past the seal between an end of the stent graft and the vessel wall, and into the aneurysmal sac, when all such flow should be contained within its lumen.

Implantable Medical Device: As used herein, “implantable medical device” includes, without limitation, stents and stent grafts used in the repair of vascular injuries.

Migration: As used herein, “migration” refers to displacement of a stent or stent graft sufficient to be associated with a complication, for example, endoleak.

Paving: As used herein, “paving” refers to a coating layer in intimate and conforming contact with a surface. The term paving in general refers to coatings in general wherein the coatings are porous or perforated or of a low porosity “sealing” variety.

Stent graft: As used herein “stent graft” shall include a fabric (or fabric and metal composite, and/or derivations and combinations of these materials) tube that reinforces a weakened portion of a vessel (in one instance, an aneurysm).

Treatment Site and Administration Site: As used herein, the phrases “treatment site” and “administration site” includes a portion of a vessel having a stent or a stent graft positioned in its vicinity. A treatment site can be, without limitation, an aneurysm site, the site of an acute traumatic aortic injury, the site of vessel narrowing or other vascular-associated pathology.

### DETAILED DESCRIPTION OF THE INVENTION

Embodiments according to the present invention include methods and devices that are useful in reducing the risk of implantable stent graft migration. More specifically, methods and devices that promote implantable stent graft attachment to blood vessel luminal walls are provided. One embodiment provides cell growth promoting factor-coated stent grafts useful for minimizing post-implantation stent graft migration following deployment at an aneurysmal treatment site and is also useful in preventing or minimizing post-implantation endoleak following stent-graft deployment at an aneurysmal treatment site.

As discussed above, an aneurysm is a swelling, or expansion of a vessel lumen at a defined point and is generally associated with a vessel wall defect. Aneurysms are often multi-factorial asymptomatic vessel diseases that if left unchecked can result in spontaneous rupture, often with fatal consequences. One method to treat aneurysms involves a highly invasive surgical procedure where the affected vessel region is removed and replaced with a synthetic graft that is sutured in place. However, this procedure is extremely risky and generally only employed in otherwise healthy vigorous patients who can be expected to survive the associated surgical trauma. Elderly and feeble patients are not candidates for these aneurysmal surgeries, and, before the development of stent grafts, remained untreated and at continued risk for sudden death.

In contrast to the described invasive surgical procedures, stent grafts can be deployed with a cut down procedure or percutaneously using minimally invasive procedures. Essentially, a catheter having a stent graft compressed and fitted into the catheter's distal tip is advanced through an artery to the aneurysmal site. The stent graft is then deployed within the vessel lumen juxtaposed to the weakened vessel wall forming an inner liner that insulates the aneurysm from the body's hemodynamic forces thereby reducing the risk of rupture. The size and shape of the stent graft is matched to the treatment site's lumen diameter and aneurysm length. Moreover, branched grafts are commonly used to treat abdominal aortic aneurysms that are located near the iliac branch.

Stent grafts generally comprise a metal scaffolding having a biocompatible covering such a Dacron® (E.I. du Pont de Nemours & Company, Wilmington, Del.) or a fabric-like material woven from a variety of biocompatible polymer fibers. Other embodiments include extruded sheaths and coverings. The scaffolding is generally on the luminal wall-contacting surface of the stent graft and directly contacts the vessel lumen. The sheath material is stitched, glued or molded onto the scaffold. In other embodiments, the scaffolding can be on the graft's blood flow contacting surface or interior. When a self-expanding stent graft is deployed from the delivery catheter, the scaffolding expands to fill the lumen and exerts circumferential force against the lumen wall. This circumferential force is generally sufficient to keep the stent-graft from migrating and thus preventing endoleak. However, stent migration and endoleak can occur in vessels that have irregular shapes or are shaped such that they exacerbate hemodynamic forces within the lumen. Stent migration refers to a stent graft moving from the original deployment site, usually in the direction of the blood flow. Endoleak (as used herein) refers specifically to the seepage of blood around the stent ends to pressurize the aneurysmal sac or between the stent graft and the lumen wall. Stent graft migration can result in the aneurysmal sac being exposed to blood pressure again and increasing the risk of rupture. Endoleaks occur in a small percentage of aneurysms treated with stent grafts. Therefore, it would be desirable to have devices, compositions and methods that minimize post implantation stent graft migration and endoleak.

Tissue in-growth and endothelialization around the stent graft have been proposed as methods to reduce the risk of stent graft migration and endoleak. Certain embodiments according to the present invention provide mechanisms to further stimulate tissue in-growth at one or more portions of a stent graft by providing a stent graft with one or more bare metal portions coated with a substance comprising a biocompatible polymer and a cell growth promoting factor on the one or more bare metal portions that promotes growth of cells from the vascular endothelium around the bare metal portions. Other embodiments according to the present invention provide mechanisms to further stimulate tissue in-growth around a stent graft by providing a a substance comprising a biocompatible polymer and a cell growth promoting factor on all or a subset of all bare metal portions found on a particular stent graft at a location other than the ends. In other embodiments, instead of or in addition to being found on bare metal portions of a stent graft, the substance comprising a biocompatible polymer and a cell growth promoting factor can be attached or woven into the material that forms the stent graft itself. As will be understood by one of skill in the art, however, and in light of further description provided herein, including the substance comprising a biocompatible polymer and a cell growth promoting factor on bare metal portions that can then be attached to the stent graft material can provide a more efficient manufacturing process than including the substance within the stent graft material itself. Both approaches, either alone or in combination, however, are included within the scope of the present invention.

Cell growth can be promoted by a variety of growth factors including, but not limited to vascular endothelial growth factor (VEGF), platelet-derived growth factor (PDGF), platelet-derived epidermal growth factor (PDEGF), fibroblast growth factors (FGFs) including acidic FGF (also known as FGF-1) and basic FGF (also known as FGF-2), transforming growth factor-beta (TGF-β), platelet-derived angiogenesis growth factor (PDAF). Cell growth can also be stimulated by induced angiogenesis, resulting in formation of new capillaries in the interstitial space and surface endothelialization, particularly by VEGF and acidic and basic fibroblast growth factors.

In one embodiment according to the present invention, the cell growth promoting factor is basic fibroblast growth factor.

The discussion of these factors is for exemplary purposes only, as those of skill in the art will recognize that numerous other growth factors have the potential to induce cell-specific endothelialization and induce cell growth. Co-pending U.S. patent application Ser. No. 10/977,545, filed Oct. 28, 2004 which is hereby incorporated by reference, discloses injecting autologous platelet gel (APG) into the aneurysmal sac and/or between an implanted stent graft and the vessel wall to induce endothelialization of the stent graft to prevent stent graft migration and resulting endoleak. Autologous platelet gel is formed from autologous platelet rich plasma (PRP) mixed with thrombin and calcium. The PRP contains a high concentration of platelets that can aggregate for plugging, as well as release high levels of cytokines, growth factors or enzymes following activation by thrombin. The development of genetically-engineered growth factors also is anticipated to yield more potent endothelial cell-specific growth factors. Additionally it may be possible to identify small molecule drugs that can induce cell growth and/or endothelialization. Thus, the stent grafts according to the present invention can improve tissue in-growth through providing substances that promote cell growth near the ends of the stent graft, or at any other point along the length of the stent graft, and in some embodiments further by providing and releasing an endothelialization factor at one or more ends or along the length of the stent graft.

In one embodiment according to the present invention, cell growth promoting factors are delivered to a treatment site within a vessel lumen associated with a stent graft. The vessel wall's blood-contacting lumen surface comprises a layer of endothelial cells. In the normal mature vessel the endothelial cells are quiescent and do not multiply. Thus, a stent graft carefully placed against the vessel wall's blood-contacting luminal surface rests against a quiescent endothelial cell layer. However, if cell growth promoting compositions are present, the normally quiescent endothelial cells lining the vessel wall, and in intimate contact with the stent graft luminal wall contacting surface, will be stimulated to proliferate. The same will occur with smooth muscle cells and fibroblasts found within the vessel wall. As these cells proliferate they will grow into and around the stent graft lining such that the stent graft becomes physically attached to the vessel lumen rather than merely resting against it.

In one embodiment of the present invention, the cell growth promoting factors are coated, or paved, onto the bare metal portions of the stent graft in a polymeric material. The basic requirements for the polymeric material to be used in the stent grafts of the present invention are biocompatibility and the capacity to be chemically or physically reconfigured under conditions which can be achieved in vivo. Such reconfiguration conditions can involve heating, cooling, mechanical deformation, (e.g., stretching), or chemical reactions such as polymerization or cross-linking.

Suitable polymeric materials for use in the invention include both biodegradable and biostable polymers and copolymers of carboxylic acids such as glycolic acid and lactic acid, polyalkylsulfones, polycarbonate polymers and copolymers, polyhydroxybutyrates, polyhydroxyvalerates and their copolymers, polyurethanes, polyesters such as poly(ethylene terephthalate), polyamides such as nylons, polyacrylonitriles, polyphosphazenes, polylactones such as polycaprolactone, polyanhydrides such as poly[bis(p-carboxyphenoxy)propane anhydride] and other polymers or copolymers such as polyethylenes, hydrocarbon copolymers, polypropylenes, polyvinylchlorides and ethylene vinyl acetates.

In one embodiment according to the present invention, suitable biocompatible and biodegradable polymers include polyglycolic acid, poly-glycolic acid/poly-L-lactic acid copolymers, polycaprolactone, polyhydroxybutyrate/hydroxyvalerate copolymers, poly-L-lactide, polydioxanone, polycarbonates, and polyanhydrides.

In one embodiment the coating, or paving, material is a homopolymer, or a binary or teriary copolymer, however, copolymers having more than three constituents are intended to be included as well.

The polymers and copolymers can sometimes contain additives such as plasticizers (e.g., citrate esters), to improve their function, such as to reduce the temperature at which sufficient fluency is obtained. In addition, physical blends of polymers including the combinations of several different biostable and/or biodegradable polymers could be utilized in this process. Likewise the process allows polymeric composites or blends of the polymers described above incorporating separate polymeric, metallic, or other, material domains to be introduced onto tissue or tissue contacting surfaces. Such domains can be present as randomly or uniformly distributed microparticles, microcapsules, nanoparticles, nanocapsules or liposomes of uniform or random size shape or compositions.

Other bioabsorbable polymers could also be used either singly or in combination. For example, homopolymers and copolymers of delta-valerolactone and p-dioxanone as well as their copolymers can be crosslinked with bis-caprolactone to provide material for use in coating the stent grafts of the present invention with cell growth promoting factors. Likewise, copolymers of polycaprolactones and lactides are also considered to be particularly useful in the present invention.

In one embodiment, the cell growth promoting stents grafts of the present invention utilize biodegradable polymers, with specific degradation characteristics to provide material having a sufficient lifespan for the particular application. As used herein, “biodegradable” is intended to describe polymers and copolymers that are non-permanent and removed by natural or imposed therapeutic biological and/or chemical processes. As such, bioerodable or bioabsorbable polymers and the like are intended to be included within the scope of that term.

The rate of bioabsorption of polycaprolactone is ideal for applications of the invention. The degradation process of this polymer has been well characterized with the primary degradation product being nontoxic 6-hydroxy hexanoic acid of low acidity. Furthermore, the time over which biodegradation of polycaprolactone occurs can be adjusted through copolymerization.

Polycaprolactone has a crystalline melting point of 60° C. and can be deployed in vivo via a myriad of techniques which facilitate transient heating and varying degrees of mechanical deformation or application as dictated by individual situations. This differs markedly from other bioabsorbable polymers such as polyglycolide and polylactide which melt at much higher temperatures (approximately 180° C.).

Polyanhydrides have been described for use as drug carrier matrices by Leong et al., J. Biomed. Mat. Res. 19, 941-955 (1985). These materials frequently have fairly low glass transition temperatures, in some cases near normal body temperature, which makes them mechanically deformable with only a minimum of localized heating. Furthermore, they offer erosion times varying from several months to several years depending on the particular polymer selected.

Heating of the polymeric material to render it fluent can be achieved using a variety of methods. For example, the polymer can be heated using a heated fluid such as hot water or saline, or it can be heated using radiofrequency energy or resistance heating. Alternatively, the polymer can be heated using light such as light having a wavelength in the infrared, visible, or ultraviolet spectrum. In still other embodiments, heating can be achieved using microwaves or radiation produced by fission or fusion processes.

The polymeric materials can be applied in custom designs, with varying thicknesses, lengths, and three-dimensional geometries (e.g. spot, stellate, linear, cylindrical, arcuate, spiral) to achieve varying finished geometries.

Further to the above, the paving coating can be applied as a continuous layer either with or without perforations. As noted earlier, in the case in which the paving coating is applied without perforations, it is referred to as a “seal” to act as a barrier layer. Such coatings can also be used to provide structural support to the stent graft, locally deliver therapeutic agents to a tissue surface, or achieve any of the other therapeutic effects, either alone or in combination, described herein. Although porous or perforated paving layers do not provide a barrier effect, each of the other aspects of the material described herein can be achieved. It is noted that as used herein the term “continuous” refers to coatings interconnected as a single unit as opposed to “discontinuous” layers which are formed of a plurality of isolated, discontinuous domains of the coating material.

The polymeric materials used in coating the cell growth promoting stent grafts of the present invention can additionally be combined with a variety of therapeutic agents for on-site delivery. Examples of such materials for use in coronary artery applications are anti-thrombotic agents, e.g., prostacyclin, heparin and salicylates, thrombolytic agents e.g. streptokinase, urokinase, tissue plasminogen activator (TPA) and anisoylated plasminogen-streptokinase activator complex (APSAC), vasodilating agents i.e. nitrates, calcium channel blocking drugs, anti-proliferative agents i.e. colchicine and alkylating agents, intercalating agents, antisense oligonucleotides, ribozymes, aptomers, growth modulating factors such as interleukins, transformation growth factor β and congeners of platelet derived growth factor, monoclonal antibodies directed against growth factors, anti-inflammatory agents, both steriodal and non-steroidal, modified extracellular matrix components or their receptors, lipid and cholesterol sequestrants and other agents which can modulate vessel tone, function, arteriosclerosis, and the healing response to vessel or organ injury post intervention. In applications where multiple polymer layers are used, different pharmacological agents could be used in different polymer layers.

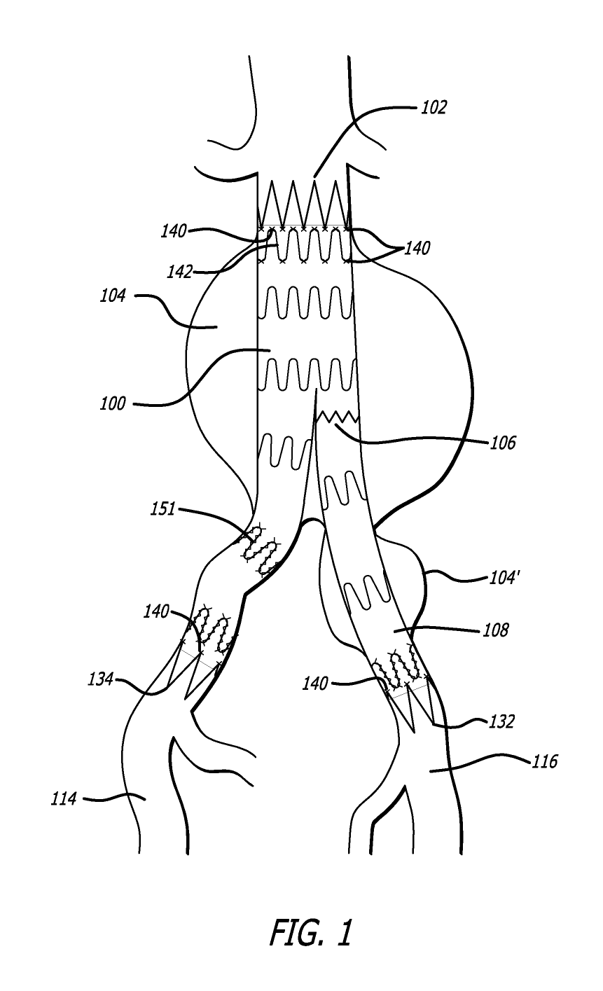

In one embodiment, a stent graft is provided “pre-loaded” into a delivery catheter. In an exemplary embodiment, a stent graft 100 is fully deployed to the site of an abdominal aortic aneurysm through the right iliac artery 114 to an aneurysm site 104 and 104′ (FIG. 1). The stent graft 100 depicted in FIG. 1 has a distal end 102 comprised of bare metal portion and an iliac leg 108 also with a bare metal portion 132 to anchor the stent graft in the left iliac artery 116. Stent graft 100 is deployed first in a first delivery catheter and the iliac leg 108 is deployed in a second delivery catheter. The stent graft 100 and iliac leg 108 are joined with a 2 cm overlap of the two segments 106. In the embodiment depicted in FIG. 1, the bare metal portions 102, 132, 134 are found at the ends of the stent graft. These bare metal portions 102, 132, 134 are attached to the stent graft 100 at connection points 140 by any appropriate method including, without limitation, by stitching. Embodiments of the present invention can also comprise bare metal portions along the length of stent graft 100 such as those depicted by, for example, bare metal portions 142 and 151. In one embodiment, bare metal portions, such as that depicted by 142, can be provided for further structural support of stent graft 100 and for release of cell growth promoting factors. As will be understood by one of ordinary skill in the art, these bare metal portions can be found on any combination, number or position on a particular stent graft. One embodiment of bare metal portions 102 and 142, and connection points 140 of stent graft 100 can be seen in more detail in FIG. 3.

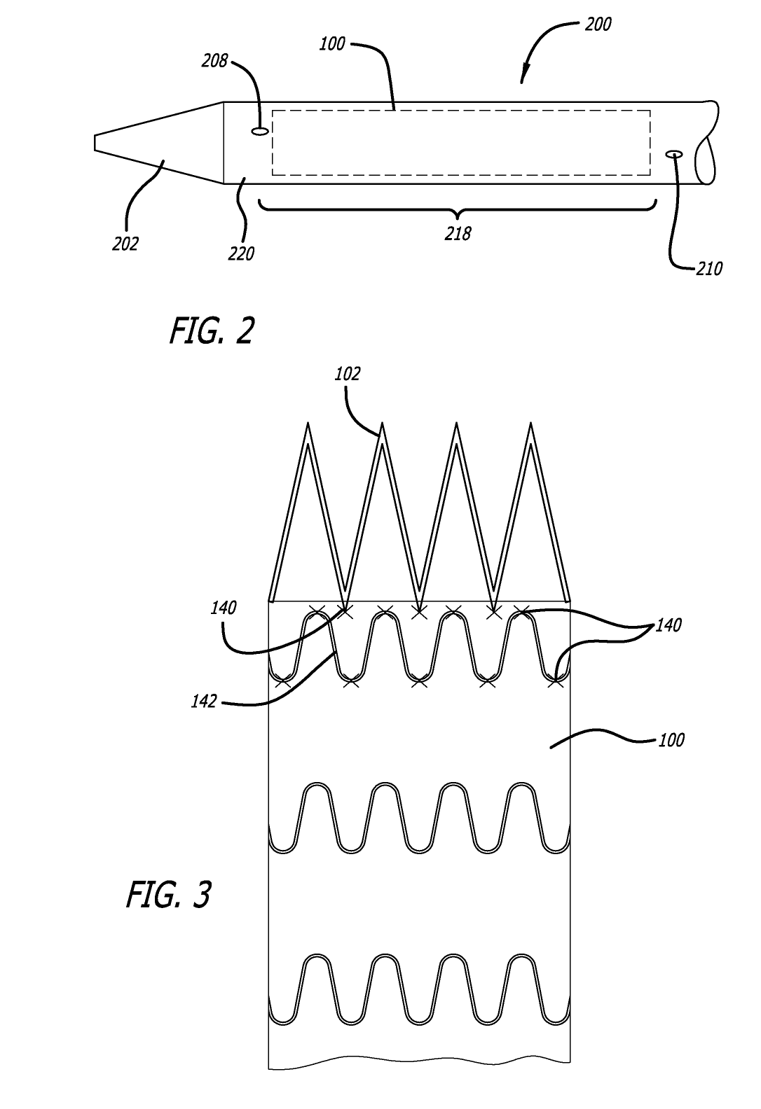

In another embodiment, a stent graft comprising a substance that promotes cell growth on one or more bare metal portions is pre-loaded into a delivery catheter such as that depicted in FIG. 2. Stent graft 100 is radially compressed to fill the stent graft chamber 218 in the distal end 202 of delivery catheter 200. The stent graft 100 is covered with a retractable sheath 220. Catheter 200 has two injection ports 208 and 210 for delivering the biocompatible polymer and cell growth promoting factor to the compressed stent graft. In this embodiment, the coating material is injected through either or both of injection ports 208 and 210 to wet stent graft 100. Stent graft 100 is then deployed to the treatment site as depicted in FIG. 1.

The field of medical device coatings is well established and methods for coating stent grafts with drugs, with or without added polymers, are well known to those of skill in the art. Non-limiting examples of coating procedures include spraying, dipping, waterfall application, heat annealing, etc. The amount of coating applied to a stent graft can vary depending upon the desired effect of the compositions contained within the coating. The coating can be applied to the entire stent graft or to a portion of the stent graft. Thus, various drug coatings applied to stent grafts are within the scope of embodiments according to the present invention.

Unless otherwise indicated, all numbers expressing quantities of ingredients, properties such as molecular weight, reaction conditions, and so forth used in the specification are to be understood as being modified in all instances by the term “about.”

Variations on embodiments will become apparent to those of ordinary skill in the art upon reading the foregoing description.

Furthermore, numerous references have been made to patents and printed publications throughout this specification. Each of the above cited references and printed publications are herein individually incorporated by reference in their entirety.

In closing, it is to be understood that the embodiments according to the invention disclosed herein are illustrative. Other modifications can be employed. Thus, by way of example, but not of limitation, alternative configurations invention can be utilized in accordance with the teachings herein.

I claim:

1. A stent graft comprising one or more exposed bare metal portions and a substance on one or more of said bare metal portions wherein said substance promotes cell growth.

2. The stent graft according to claim 1, wherein at least one of said bare metal portions is found at the end of said stent graft.

3. The stent graft according to claim 1, wherein said substance comprises a biocompatible polymer and a cell growth promoting factor.

4. The stent graft according to claim 3, wherein said biocompatible polymer is biodegradable.

5. The stent graft according to claim 4, wherein said biocompatible and biodegradable polymer is selected from the group consisting of polyglycolic acid, poly˜glycolic acid/poly-L-lactic acid copolymers, polycaprolactone, polyhydroxybutyrate/hydroxyvalerate copolymers, poly-L-lactide, polydioxanone, polycarbonates, and polyanhydrides.

6. The stent graft according to claim 3, wherein said cell growth promoting factor is basic fibroblast growth factor.

7. A method for treating an aneurysm comprising:
providing a stent graft comprising one or more exposed bare metal portions and a substance on one or more of said bare metal portions wherein said substance promotes cell growth.

8. The method according to claim 7, wherein at least one of said provided bare metal portions is found at the end of said stent graft.

9. The method according to claim 7, wherein said substance comprises a biocompatible polymer and a cell growth promoting factor.

10. The method according to claim 9, wherein said biocompatible polymer is biodegradable.

11. The method according to claim 10, wherein said biocompatible and biodegradable polymer is selected from the group consisting of polyglycolic acid, poly˜glycolic acid/poly-L-lactic acid copolymers, polycaprolactone, polyhydroxybutyrate/hydroxyvalerate copolymers, poly-L-lactide, polydioxanone, polycarbonates, and polyanhydrides.

12. The stent graft according to claim 9, wherein said cell growth promoting factor is basic fibroblast growth factor.

13. A method comprising:
providing a stent graft comprising one or more exposed bare metal ends and a substance on one or more of said bare metal ends wherein said substance promotes cell growth, is in the form of a polymeric material comprising a cell growth promoting factor; wherein said cell growth promoting factor comprises basic fibroblast growth factor; said polymeric material is selected from the group consisting of polyglycolic acid, poly˜glycolic acid/poly-L-lactic acid copolymers, polycaprolactone, polyhydroxybutyrate/hydroxyvalerate copolymers, poly-L-lactide, polydioxanone, polycarbonates, polyanhydrides; and
positioning said stent graft at a treatment site wherein said substance contributes to the fixation of said stent graft to the vessel wall at said treatment site.

14. A method according to claim 13, wherein said treatment site is an aneurysm site.
